(19) United States
(12) Patent Application Publication
(10) Pub. No.: US 2009/0029956 A1
(43) Pub. Date: Jan. 29, 2009

(54) Substance mixture

(76) Inventors: Bruno Pregenzer, Mieming (AT); Alfred Konzett, Patsch (AT); Andreas Neher, Innsbruck (AT); Waldemar Gottardi, Innsbruck (AT); Markus Nagl, Axams (AT)

Correspondence Address: CARTER, DELUCA, FARRELL & SCHMIDT, LLP, 445 BROAD HOLLOW ROAD, SUITE 420, MELVILLE, NY, 11747, US

(21) Appl. No.: 11/795,220

(22) PCT Filed: Jan. 12, 2006
(86) PCT No.: PCT/AT06/00011

§ 371 Date: Apr. 30, 2008

(30) Foreign Application Priority Data
Jan. 17, 2005 (AT) A 66/2005

(51) Int. Cl. A61K 31/573; A61P 29/00

(52) U.S. Cl. 514/171

(57) ABSTRACT

A substance mixture comprising at least one glucocorticoid and at least one N-chloro compound and/or at least one O-chloro compound as well as a medicament containing such a substance mixture as well as the use and manufacture of such a medicament for the topical treatment of inflammations.

The invention relates to a substance mixture containing at least one glucocorticoid and at least one N-chloro compound and/or at least one O-chloro compound, to a medicament containing this substance mixture as well as to the use of this medicament for the topical treatment of inflammations, in particular of acute inflammations.

The treating physician selects different forms of treatment for the therapy of the complaint depending on the course of an inflammatory complaint. The use of glucocorticoids, or so-called cortisone preparations, is usual for chronic and long-term inflammations since cortisone preparations have good anti-inflammatory properties.

The treatment of acute inflammatory complaints with cortisone preparations is avoided since glucocorticoids show unwanted side effects at high doses. For example, glucocorticoids have a slightly immunosuppressive effect which promotes secondary infections or diseases. Furthermore, glucocorticoids, in particular those for topical treatment, contain preservatives or disinfectants such as benzalkonium chloride which likewise have very strong side effects; they are in particular toxic to cilia; they frequently also cause toxic contact dermatitis.

Antibiotics are usually used for the treatment of inflammations, for example acute bacterial inflammations, which kill off bacteria or inhibit them in the metabolism due to their bactericidal or bacteriostatic effect. For example, practically every acute inflammation of the middle ear is generally treated with antibiotics, even if the inflammation was originally caused by viruses, because bacteria settle at the center of inflammation. It is disadvantageous in such a treatment that an antibiotic does not have any anti-inflammatory property and that negative concomitant diseases frequently occur, for example fungal infestation.

It is therefore the object of the present invention to develop a substance mixture for the preparation of a medicament for the improved topical treatment of inflammatory diseases, in particular for the treatment of acute inflammatory diseases in the auditory canal, with lower side effects.

This is achieved in accordance with the invention by the use of a substance mixture containing at least one glucocorticoid and at least one N-chloro compound and/or at least one O-chloro compound.

The use of such a substance mixture in particular has the advantage that, due to the oxidative effect of the N-chloro compound and/or of the O-chloro compound, the use of disinfectants such as benzalkonium chloride which can frequently themselves cause inflammatory diseases is avoided. Glucocorticoids require the addition of a disinfectant or preservative which has a slightly toxic effect as a rule so that the glucocorticoid remains stable in its galenic form. An advantage of the invention includes the fact e.g. that the favorable properties of the glucocorticoid (e.g. anti-inflammatory) develop their full effect without the addition of toxic additives thanks to the active ingredient combination in accordance with the invention since the combination active ingredient(s) itself/themselves acts/act as a disinfectant, with a simultaneous reduction in the risk of a secondary infection due to the effects of the N-chloro compound and/or of the O-chloro compound.

Furthermore, a substance mixture in accordance with the invention also presents itself for the treatment of any present multi-infection (e.g. a viral infection and a bacterial infection) which frequently develop due to the weakening of the immune system due to the primary infection even before the start of treatment.

A further advantage in accordance with the invention of such a substance mixture also lies in the fact that compound classes of the type of N-chloro compounds and/or O-chloro compounds themselves additionally have a bactericidal, fungicidal, virucidal and/or vermicidal effect due to the oxidative properties of these compound types. Natural glucocorticoids (e.g. cortisol, cortisone, etc.) present themselves, as glucocorticoids or cortisone preparations, on the one hand; and, on the other hand, synthetic or semi-synthetic glucocorticoids (e.g. prednisone, prednisolone, betametasone, etc.). The choice of the glucocorticoid is naturally not restricted to the named examples, but naturally includes other glucocorticoids with anti-inflammatory properties familiar to the person of ordinary skill in the art.

N-chloro derivatives of amines and amides present themselves as an N-chloro compound; in particular N-chloro amino acids, chloro amino T (N-chloro-4-toluene sulfonic acid amide sodium), chloro amine-B (N-chloro-4-benzene-sulfonic acid amide sodium), chloro isocyanuric acid, dichloro hydantoin prove favorable, with typically carboxylic acids, sulfonic acids and phosphoric acids, but also other acid types being used as N-chloro amino acids. The use of N-chlorotaurine has proved to be particularly favorable. N-chlorotaurine is a water-soluble N-chloro amino acid, specifically an N-chloro amino sulfonic acid having the formula ClHN—CH₂—CH₂—SO₃H₁, with the deprotonated acid and in particular salts, for example alkali salts or alkaline earth sales such as the sodium salt or the calcium salt, however, also being subsumed under the term N-chlorotaurine and also being used as such in accordance with the invention. In the following, both the free acid and the acid anion as well as all salts of N-chlorotaurine will therefore be subsumed under N-chlorotaurine. Examples for corresponding alkaline compounds of N-chlorotaurine are described in DE 40 41 703 C2. N-chlorotaurine is a substance which is released by stimulated human granulocytes and monocytes as part of anti-infective defenses and which has antibiotic (bactericidal), fungicidal, virucidal and vermicidal properties.

Hypochlorous acid and compounds derived from hypochlorous acid can be used as the O-chloro compound, for example alkali salts and alkaline earth salts of hypochlorous acid, in particular NaOCl, Ca(OCl)₂.

Provision is made in accordance with the invention for the substance mixture to contain 5-50% glucocorticoid (all percentage figures are percent by weight), 0-90% N-chloro compound and 0-90% O-chloro compound. It is favorable for the mixture to contain 20-40% glucocorticoid and 50-75% N-chloro compound and 0-50% O-chloro compound. A composition of 30-35% glucocorticoid and 65-70% N-chloro compound and 0-5% O-chloro compound has proved to be particularly favorable. In an embodiment, a mixture of approximately ⅓ prednisolone and approximately ⅔ N-chlorotaurine was used which has exceptional properties in accordance with the invention.

It is decisive both for the N-chloro compound and for the O-chloro compound that it is a non-toxic oxidizingly active substance. Other compounds of these compound types can therefore also be considered.

It is favorable to configure the substance mixture in accordance with the invention as a medicament. It is particularly favorable to configure the medicament in a galenic form known to the skilled person for topical application. Aqueous forms of presentation for topical application, for example, can in particular be considered for this purpose, for example in the form of solutions or suspensions which are applied as drops. The concentration favorably amounts to approximately 0-10% glucocorticoid, 0-10% N-chloro compound and 0-10% O-chloro compound. A concentration of 0.5-1% glucocorticoid, of 0-1% N-chloro compound and of 0-1% O-chloro compound proves to be particularly favorable.

Alternatively, the medicament is feasible in the galenic form of an ointment (water-free mixture), of a cream (H₂O in oil or oil in a H₂O mixture), of an emulsion, of a gel (of a liquid in which the active ingredient has been dissolved and has been mixed with a gelling agent), of a paste or of a transdermal therapeutic system (e.g. a plaster containing an active ingredient) or in particular of a suspension. A use of strips which have been soaked in a solution, a suspension or an emulsion, in a manner known per se, also presents itself for topical treatment. For example, a strip soaked in a suspension in accordance with the invention can be introduced into the auditory canal of a patient suffering from an inflammation of the auditory canal.

In all named galenic forms of medicaments, an addition of adjuvants, stabilizers, disinfectants, etc.—as usual in such forms of medicaments per se—is naturally provided within the framework of the invention. Since glucocorticoids are as a rule slightly soluble or difficult to dissolve in water, it is generally feasible, especially with aqueous forms of presentation, to add additional solubilizers or dispersants to distribute the glucocorticoid better in the solution or suspension. However, it has been shown in practical application that a complete blending in the aqueous phase is not necessarily required for successful treatment. Multi-phase systems also have the same favorable therapeutic properties.

Medicaments of this type present themselves for the treatment of topical inflammations, in particular for the treatment of acute topical inflammations. For example, the use of such a medicament has proved to be particularly favorable in the treatment of inflammations of the auditory canal.

The use of a formula in accordance with the invention will be shown for three case studies.

Patients were treated locally with an N-chlorotaurine/ glucocorticoid mixture (1% N-chlorotaurine sodium, 0.5% prednisolone in distilled water) at the Ear, Nose and Throat Clinic in Innsbruck. The patients suffered from a therapy-resistant otitis externa (inflammation of the auditory canal), from an otitis media (inflammation of the middle ear) with a perforation of the eardrum (myringotomy tubes) or from a cholesteatome with perforation of the eardrum. The therapy took place by local flushing of the auditory canal or, in the presence of a perforation of the eardrum by flushing of the middle ear, likewise via the outer auditory canal.

Case 1:

43-year old patient, suffering from an otitis externa for 18 days. He has been treated locally with Otosporin Ear Drops during this period. No improvement in symptoms occurred with this therapy. The patient is subsequently treated with strips soaked in N-chlorotaurine prednisolone and subsequently treated with N-chlorotaurine prednisolone ear drops for one day. His inflammation of the auditory canal at both sides abated after 3 days.

Case 2:

31-year old patient, suffering from a perforation of the eardrum. Fostered by this, an inflammation of the middle ear and of the outer auditory canal results. This patient is treated locally with Ciproxin Ear Drops and Clavamox (2×1 gram/day). Under this therapy, an additional fungal infestation of the auditory canal and of the middle ear arises after 4 days. The therapy is switched to a local therapy with N-chlorotaurine prednisolone. After 4 days, both the middle ear and the auditory canal are free of inflammation and the eardrum can be closed by an operation.

Case 3:

23-year old, suffering from a cholesteatome with a right eardrum perforation. The cholesteatome leads to a permanent inflammation of the outer auditory canal with permanent suppurating otorrhea. Under the therapy with N-chlorotaurine prednisolone ear drops, the auditory canal remains free of inflammation and dry up to the operation.

The combination preparation in accordance with the invention therefore has excellent properties in the treatment of inflammations of the auditory canal. Due to the low dose of glucocorticoid dispensed, no side effects typical for glucocorticoids resulted for the patient (e.g. muscle atrophy, osteoporosis, increase in blood glucose, skin atrophy, moon face, problems with the crystalline lens, growth disorders and immunodeficiency). A main advantage of the invention lies in the fact that no preservatives or disinfectants such as benzalkonium chloride have to be used since these preservatives or disinfectants frequently have strong side effects. Furthermore, N-chlorotaurine proves to be an ideal combination with the anti-inflammatory glucocorticoid due to its antibiotic effect. No resistance to N-chlorotaurine develops since it is a substance occurring in the body and is therefore also very compatible. No further preservative is needed due to the oxidative effect.

The application for other inflammations which are open to local therapy using this combination preparation also presents itself provided that the therapeutic agent can be introduced to the point of inflammation in a sufficient quantity without systemic application.

The favorable effect of the combination preparation was demonstrated with reference to the regeneration capability of human ciliary cells. In comparison with this, the seriously damaging effect of preservatives (on the basis of benzalkonium chloride, BAC) used in conventional cortisone preparations on human ciliary cells was compared. The flicker frequency was determined in a manner known per se by means of a photometric method. Solutions and fluticasone propionate and mixed solutions of fluticasone propionate with N-chlorotaurine sodium (NCT) were prepared in accordance with the following table:

| | | Fluticasone |
|---|---|---|
| Solution: | NCT | propionate: |
| A | — | 0.5 mg/ml |
| B | — | 0.05 mg/ml |
| C | 10 mg/ml | 0.5 mg/ml |

Mucous membrane cells were first incubated with an isotonic NaCl solution for 20 minutes (Time I), subsequently incubated with the solutions A, B and C for 20 minutes (Time II) and subsequently incubated in an isotonic solution for a further 20 minutes (Time III). Parallel to this, epithelian mucous membrane cells were incubated according to the same pattern in comparison with BAC solutions (concentrations of 0.04 mg/ml; 0.1 mg/ml; 0.2 mg/ml; 0.5 mg/ml). The results of the regeneration capability of the ciliary cells after treatment with the solutions A, B and C were compared with the results after the treatment with the BAC solutions. These results are collated in the following table.

Mean value in percent

| | 20 min NaCl | 20 min test | 20 min NaCl |
|---|---|---|---|
| Test solution | 0.9% (I) | Solution: (II) | 0.9% (III) |
| C | 100 | 42.2 | 68.9 |
| A | 100 | 33.9 | 68.8 |
| B | 100 | 55.2 | 73.5 |
| 0.04 mg/ml BAC | 100 | 96.6 | 100 |
| 0.1 mg/ml BAC | 100 | 91.9 | 93.6 |
| 0.2 mg/ml BAC | 100 | 63.5 | 19.4 |
| 0.5 mg/ml BAC | 100 | 0 | 0 |

As can be recognized from the table, ciliary cells recover clearly again after a treatment with even a 1% solution of N-chlorotaurine with fluticasone propionate (test solution C) after 20 minutes, whereas the ciliary cells remain permanently damaged and regeneration no longer takes place after a treatment with more than 0.1% mg/ml benzylalkonium chloride (0.1%).

1. A composition comprising at least one glucocorticoid and at least one, compound selected from the group consisting of an N-chloro compound and an O-chloro compound.

2. A composition in accordance with claim 1, wherein the glucocorticoid is a natural, synthetic or semi-synthetic glucocorticoid.

3. A composition in accordance with claim 1, wherein the N-chloro compound is selected from the group consisting of N-chloro derivates of amines and amides.

4. A composition in accordance with claim 1, wherein the N-chloro compound is N-chlorotaurine or a salt.

5. A composition in accordance with claim 1, wherein the O-chloro compound is a hypochlorous acid or an alkali salt or an alkaline earth salt of hypochlorous acid.

6. A composition in accordance with claim 1, wherein the composition contains 5-50% glucocorticoid and 0-90% N-chloro compound and 0-90% O-chloro compound, preferably 20-40% glucocorticoid and 50-75% N-chloro compound and 0-50% O-chloro compound and particularly favorably 30-35% glucocorticoid and 65-70% N-chloro compound and 0-50% O-chloro compound.

7. A composition in accordance with claim 1, which comprises up to approximately ⅓ prednisolone and up to approximately ⅔ N-chlorotaurine and/or a salt of N-chlorotaurine.

8. A medicament containing a composition in accordance with claim 1.

9. A medicament in accordance with claim 8, wherein the medicament is adapted to be applied in a galenic form for topical application.

10. A medicament in accordance with claim 9, wherein the galenic form is an aqueous form, preferably a solution or suspension.

11. A medicament in accordance with claim 10, wherein the aqueous form comprises concentration of 0.1-10% glucocorticoid and of 0-10% N-chloro compound and of 0-10% O-chloro compound, but preferably concentrations of 0.2-1.5% glucocorticoid and of 0-2% N-chloro compound and of 0-2% O-chloro compound.

12. A medicament in accordance with claim 9, wherein the galenic form is selected from the group consisting of an ointment, a cream, an emulsion, a gel, a paste, a suspension, drops and a strip soaked in a solution or suspension.

13. Use of a composition in accordance with claim 1 for the manufacture of a medicament for the topical treatment of inflammations.

14. Use in accordance with claim 13 for the treatment of acute inflammations.

15. Use in accordance with claim 13 for the treatment of inflammations of the auditory canal.

16. A composition in accordance with claim 2, wherein the glucocorticoid is selected from the group consisting of cortisone, hydrocortisone, predisone and prednisolone.

17. A composition in accordance with claim 3, wherein the N-chloro compound is selected from the group consisting of N-chloro amino acids, chloro amino T, chloro amino B, chloro isocyanuric acids and hydantoin.

18. A composition in accordance with claim 4, wherein the salt of the N-chloro compound is an alkaline earth salt of N-chlorotaurine.

19. A composition in accordance with claim 5, wherein O-chloro compound is selected from the group consisting of sodium hypochloride and calcium hypochoride.

20. A method of treating topical inflammation in a patient comprising administering a composition according to claim 1 in an amount effective to treat topical inflammation.
